(19) United States
(12) Patent Application Publication
(10) Pub. No.: US 2026/0165864 A1
(43) Pub. Date: Jun. 18, 2026

(54) SYSTEM AND METHOD FOR PREVENTION OR CORRECTION OF INFANT CRANIAL SHAPE DEFORMITIES

(71) Applicant: Gary ROGERS, Great Falls, VA (US)

(72) Inventor: Gary ROGERS, Great Falls, VA (US)

(73) Assignee: Gary ROGERS, Great Falls, VA (US)

(21) Appl. No.: 18/979,257

(22) Filed: Dec. 12, 2024

(51) Int. Cl. A61F 5/01

(52) U.S. Cl. CPC A61F 5/01

(57) ABSTRACT

An infant orthotic system includes a base including a main surface elevated off a ground plane by a height via a skirt flanking the main surface, the main surface including a depression disposed at a first end of the main surface, the depression defined by an opening in the main surface, the main surface configured to support the infant disposed thereon, the depression configured to receive a head of the infant disposed therein; and an insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant.

### BACKGROUND

### Field of the Disclosure

This disclosure relates to a system for preventing or correcting deformational posterior plagiocephaly.

### Description of the Related Art

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Plagiocephaly typically progresses most in infants ranging between birth and 3-4 months of age. Deformational posterior plagiocephaly is a localized flattening of the cranium, usually near the occiput.

Deformational posterior plagiocephaly occurs most often in infants of four months age or younger because (i) the skulls of young infants are still easily deformed; (ii) the posterior calvaria are rapidly expanding against a flat surface; and (iii) such infants are neuromuscularly immature and lack the coordination to shift their head position. If left uncorrected, the pressure forces will likely remain continuously applied until the infant reaches 6-8 months of age, whereupon the existing deformation will likely become permanent.

As described in U.S. Pat. No. 8,758,283, which is incorporated herein by reference in its entirety for all purposes, an orthotic device can help correct or prevent plagiocephaly using a bed, pad, or mattress with multiply layered material inserts that are arranged and aligned in overlay series (a nested stack). In turn, as the child grows, an inset layer can be removed. While the multiple material inserts in the nested stack can be pre-designed to fit for most average infant head dimensions, the issue of plagiocephaly, which can present uniquely per infant, can benefit greatly from more customized or individualized head inserts that can be replaced entirely.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

The present disclosure relates to an infant orthotic system, including a base including a main surface elevated off a ground plane by a height via a skirt flanking the main surface, the main surface including a depression disposed at a first end of the main surface, the depression defined by an opening in the main surface, the main surface configured to support the infant disposed thereon, the depression configured to receive a head of the infant disposed therein; and an insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust to the shape and size of the infant's head.

The present disclosure additionally relates to a method for preventing and correcting cranial shape deformities, the method including providing a base including a main surface elevated off a ground plane by a height via a skirt flanking the main surface, the main surface including a depression disposed at a first end of the main surface, the depression defined by an opening in the main surface, the main surface configured to support an infant disposed thereon, the depression configured to receive a head of the infant disposed therein; generating an insert, the insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant; arranging the insert in the depression; and arranging the infant on the base, a body of the infant being disposed on the main surface and a head of the infant being disposed in the insert.

The present disclosure additionally relates to a non-transitory computer-readable storage medium for storing computer-readable instructions that, when executed by a computer, cause the computer to perform a method, the method including: receiving obtained images of a head of an infant; determining a head shape of the infant based on the obtained images; determining a deviation between the determined head shape of the infant and a reference head shape for a reference infant; determining an interior shape of a concave portion of an insert configured to receive the head of the infant disposed therein that mitigates the deviation between the determined head shape of the infant and the reference head shape for the reference infant; and generating the insert with the determined interior shape, the insert including the concave portion and a flange surrounding the concave portion, the flange extending outwards from an edge of the concave portion, a depression of a base configured to receive the insert inserted therein, the flange configured to rest on an edge of the depression, the interior shape of the concave portion configured to adjust a shape and size of the head of the infant, the base including a main surface elevated off a ground plane by a height via a skirt flanking the main surface, the main surface including the depression disposed at a first end of the main surface, the depression defined by an opening in the main surface, the main surface configured to support the infant disposed thereon.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows a schematic of a system for correcting infant cranial shape deformities and preventing plagiocephaly, according to an embodiment of the present disclosure.

FIGS. 2A-2C show schematics of a portion of the system at various perspectives, according to an embodiment of the present disclosure.

FIGS. 3A and 3B show schematics of the set of the inserts, according to an embodiment of the present disclosure.

FIG. 4A is a flow chart for a method of preventing and correcting cranial shape deformities, according to an embodiment of the present disclosure.

FIG. 4B is a flow chart for a sub-method describing step S410, according to an embodiment of the present disclosure.

FIG. 4C is a flow chart for a method 401 of preventing and correcting cranial shape deformities, according to an embodiment of the present disclosure.

FIG. 5 is a schematic view of user devices communicatively connected to a server, according to an embodiment of the present disclosure.

FIG. 6 is a block diagram illustrating an electronic user device, according to an embodiment of the present disclosure.

FIG. 7 is a schematic of a hardware system for performing a method, according to an embodiment of the present disclosure.

FIG. 8 is a schematic of a hardware configuration of a device for performing a method, according to an embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

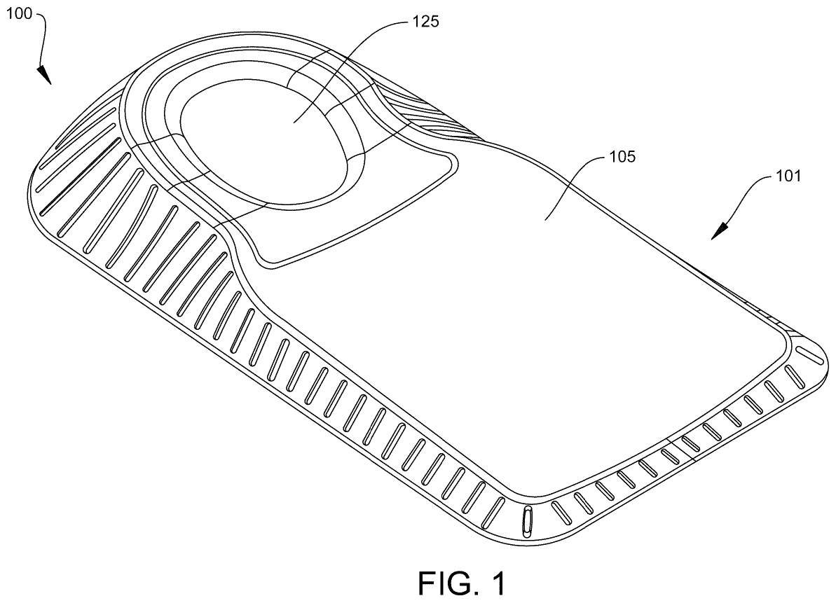

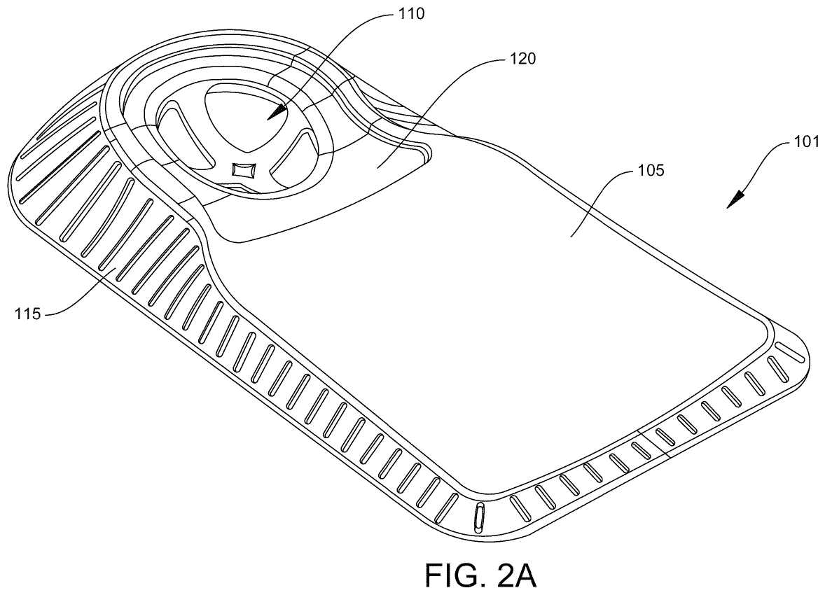

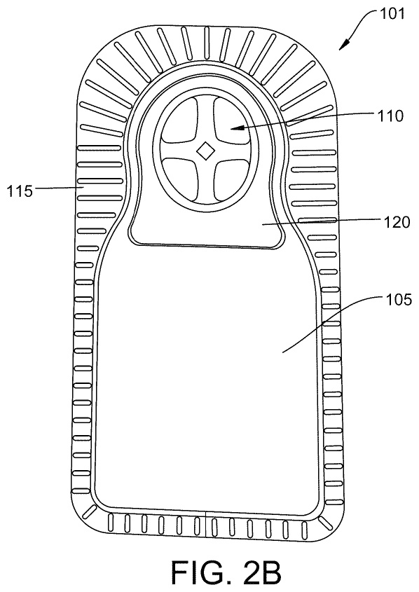

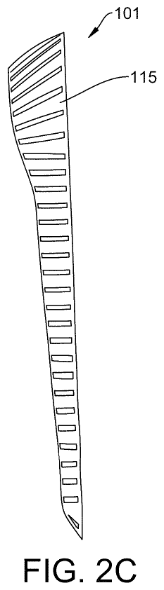

FIG. 1 shows a schematic of a system 100 for correcting infant cranial shape deformities and preventing plagiocephaly, according to an embodiment of the present disclosure. FIGS. 2A-2C show schematics of a portion of the system 100 at various perspectives, according to an embodiment of the present disclosure. In an embodiment, the system 100 includes a base 101, and the base 101 can include a main surface 105, a depression 110, and a skirt 115. The base 101 can be configured to support an infant disposed thereon arranged in a supine position. In such an arrangement, a body of the infant (e.g., torso with arms and lower body with legs) can be disposed on the main surface 105 of the base 101, while a head of the infant can be disposed on or in the depression 110. Thus, the main surface 105 can be referred to as a sleeping surface, supporting surface, laying surface, main surface, or the like. As shown, the main surface 105 can be elevated off of an underlying surface via the skirt 115. The skirt 115 can be configured to provide the elevation off the underlying surface in order to allow the depression 110 to have a depth.

In an embodiment, the main surface 105 can be level with the underlying surface or angled relative to the underlying surface. When the main surface 105 is angled relative to the underlying surface, the main surface 105 can have an incline that starts from where feet of the infant would be arranged and increases towards where shoulders or the head of the infant would be arranged on the main surface 105. An angle of the incline can be, for example, more than 0° and less than 15°, or 0.5° to 10°, or 1° to 5°, or 2° to 4.5°, or 3° to 4°, or 3.5°, or 3.6°.

In an embodiment, the main surface 105 can be rectangular in shape where the infant's body can be disposed and narrow to a rounder shape where the infant's head can be disposed (as shown). Additionally or alternatively, the main surface 105 and the area of the depression 110 can both entirely form a rectangular shape with no narrowing towards the depression 110.

In an embodiment, the skirt 115 can be a peripheral skirt of material around an edge of the base 101. The skirt 115 can be solid or perforated (as shown) to allow additional airflow under the base 101 and the depression 110. Notably, where the skirt 115 meets the edge of the main surface 105 can form a ridge.

In an embodiment, the main surface 105 can be substantially planar and the ridge formed is a corner with no bead or protrusion above a plane of the planar main surface 105.

In an embodiment, the main surface 105 can be curved or concave in towards the underlying surface. With such a concavity, the infant disposed therein can be more centered and the concavity can provide additional resistance to any rolling motion of the infant to go from the supine position to a prone position or position on a stomach of the infant. Furthermore, the rolling resistance due to the concavity can prevent the infant from rolling off of the main surface 105.

In an embodiment, the main surface 105 can be concave while also being sunken relative to the ridge. Thus, the ridge can have a height that helps prevent the infant from translating or rolling off of the main surface 105. The ridge can also have a varying height based on a location around the main surface 105 and the depression 110. For example, the height of the ridge towards where the infant's feet can be disposed can be very high, such as the entire elevation distance of the main surface 105 off of the underlying surface (i.e., a thickness of the skirt). This can help prevent the infant from sliding or slipping down away from the depression 110 area. For example, the height of the ridge towards where the infant's head can be disposed can be very low since the risk of slipping of rolling over in the depression 110 area is low. The concavity can also be shaped to contour to a shape of the infant. For example, an area near the shoulders of the infant can narrow towards the depression 110 where a neck of the infant can be disposed.

In an embodiment, the main surface 105 can be solid or perforated. The perforations can allow for airflow and better thermal regulation of the infant. The perforations can also allow any fluids to pass through and not pool on or around the infant, such as excessive bodily fluids released by the infant.

In an embodiment, the base 101 can have an overall length and width. For example, the length of the base 101 can be 20 to 40 inches, or 25 to 35 inches, or 28 to 32 inches, or 29 inches, or 30 inches, or 31 inches. For example, the width of the base 101 can be, for example, 10 to 20 inches, or 12 to 18 inches, or 13 to 17 inches, or 14 to 16 inches, or 14 inches, or 14.5 inches, or 15 inches.

In an embodiment, an underside of the main surface 105 and the depression 110 along with and an interior of the skirt 115 can form a chamber. The chamber can be substantially empty and allow the main surface 105 and the depression 110 to flex and elastically deform. Additionally or alternatively, the chamber can include trusses or supports spanning between opposite sides of the skirt 115 and the underside of the main surface 105 and the depression 110 in any combination. For example, the trusses, can only span between opposite sides of the skirt 115. For example, the trusses can only extend down from the underside of the main surface 105 and the depression 110. For example, the trusses extending down from the underside of the main surface 105 and the depression 110 can be connected to the trusses spanning between opposite sides of the skirt 115. The trusses can provide additional strength and rigidity to the base 101 in order to support a weight of the infant disposed on the main surface 105.

In an embodiment, the depression 110 can be an opening or a cavity disposed adjacent to the main surface 105. The depression 110 can be additionally disposed in a sunken area relative to the plane of the main surface 105, which can be referred to as an inset portion 120. As will be described below, the inset portion 120 can be sunken and configured to receive an object, such as an insert 125. Thus, the shape of the inset portion 120 can be based on a predetermined shape, such as a shape of the insert 125. Returning to the depression 110, the depression 110 can be configured to receive the insert 125. In an embodiment, the depression 110 can include additional material outlining or defining edges of the cavity, which can be configured to further support the insert 125.

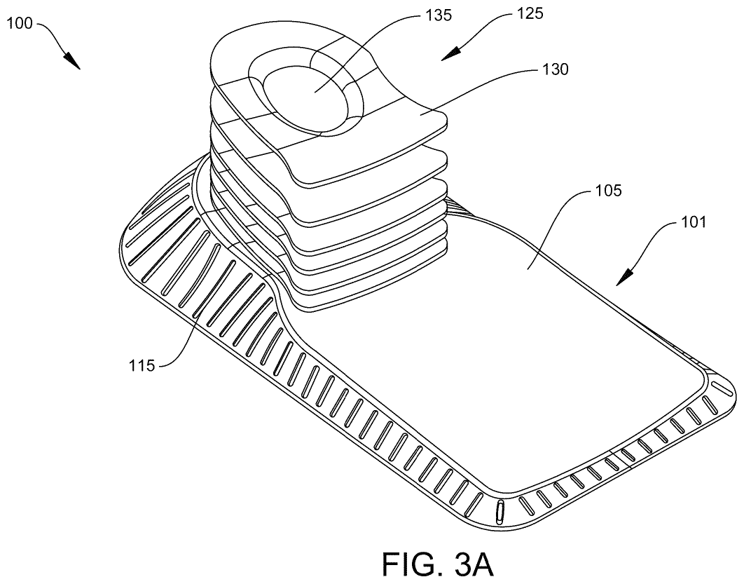

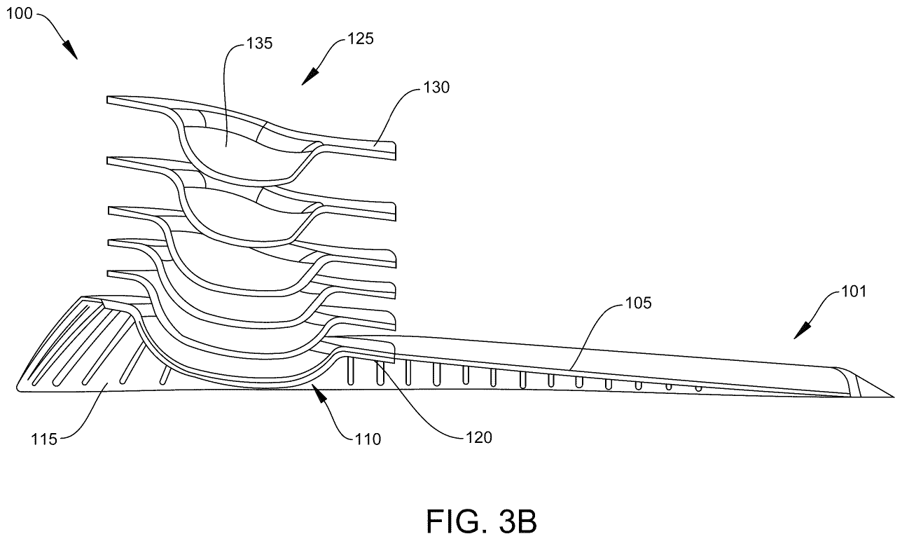

FIGS. 3A and 3B show schematics of the set of the inserts 125, according to an embodiment of the present disclosure. In an embodiment, the system 100 can include the insert 125. The insert 125 can include a flange 130 and a bowl 135. The bowl 135 can be a concave portion of the insert 125, and be similarly referred to herein as “a concave portion.” The bowl 135 (an interior shape of the bowl 135) can be shaped like an occipital region of a normal infant cranium. The flange 130 can be flared material extending from an edge of the bowl 135. The flange 130 can have a corresponding shape to the shape of the inset portion 120. Thus, the insert 125 can be disposed in the depression 110 and the flange 130 can be disposed in the inset portion 120. That is, the flange 130 of the insert 125 can rest on the sunken material in the inset portion 120 such that a top surface of the flange 130 of the insert 125 is flush with the main surface 105 proximal to the depression 110. As previously described, the depression 110 can include additional material outlining or defining edges of the cavity, which can be configured to support the insert 125. Thus, an exterior shape of the bowl 135 can abut and rest on the additional material outlining the edges of the cavity of the depression 110. Since the interior shape of the bowl 135 can vary (based on the infant, developmental stage, etc. described below), the exterior shape of the bowl 135 can be consistent across multiple inserts in order for the additional material outlining the edges of the cavity of the depression 110 to support the bowl 135.

In an embodiment, a material of the insert 125 can be sufficiently rigid to support a weight of the head of the infant. For example, the material can be a polymer, a composite, a metal, or a foam, among others. Further, the bowl 135 of the insert 125 can include padding or similar feature to improve a comfort of the insert 125 to the infant. For example, the bowl 135 can include a liner of a softer, deformable material, such as foam. For example, the bowl 135 can include a cushion or pad at predetermined positions along the bowl 135. The predetermined positions can be, for example, common contact points between the bowl 135 and the head of the infant. In an embodiment, when the depression 110 includes the additional material outlining the edges of the cavity of the depression 110, the material of the bowl 135 can be weaker and more deformable since the additional material can be the main means of supporting the weight of the infant's head.

In an embodiment, the insert 125 can be configured to receive and support the head of the infant disposed on the main surface 105 in a supine position. Due to the rapid growth and changes that occur in the early stages of an infant's life, one design for the insert 125 can quickly become outdated and non-optimal for the infant. Therefore, multiple of the insert 125 can be used and customized for the infant based on the infant's predicted development.

To this end, in an embodiment, the insert 125 can be modular and the system 100 can include a set of the inserts 125. Notably, each insert of the set of the inserts 125 can be swapped with one another based on a developmental stage of the infant. As previously described, each insert of the set of the inserts 125 can include a flange 130 and a bowl 135. Here, the flange 130 can be the same shape and dimensions across the set of the inserts 125, while the bowl 135 can be different. The bowl 135 for each insert of the set of the inserts 125 can have a different interior shape based on the developmental stage of the infant. For example, a first insert of the set of the inserts 125 can be shaped like an occipital region of a newborn infant cranium, a second insert of the set of the inserts 125 can be shaped like an occipital region of a 1- to 2-month old infant cranium, a third insert of the set of the inserts 125 can be shaped like an occipital region of a 3- to 4-month old infant cranium, and a fourth insert of the set of the inserts 125 can be shaped like an occipital region of a 5- to 6-month old infant cranium. Each insert can progressively enlarge and change contour. Thus, as the infant ages, a caregiver for the infant can remove an insert that is no longer recommended and swap in an insert that corresponds to the updated developmental stage of the infant.

In one embodiment, since the infant's head is the only part of the infant's body that is at risk of a shape issue (i.e., plagiocephaly), the insert 125 can advantageously be the only portion of the system 100 that needs to be swapped and the remainder of the system 100 (e.g., the base 101, additional padding, linens, etc.) can remain the same. The modular system having swappable inserts can also make it easier to replace the insert 125 with another since each insert of the set of the inserts 125 has the same shape and dimensions for the flange 130 and the same exterior shape for the bowl 135.

Furthermore, the modular insert 125 can allow for highly customized interior shapes for the bowl 135. While a standard interior shape for the bowl 135 across multiple of the insert 125 can be used (e.g., a “one-size-fits-most” interior shape progression for a set of the inserts), customized sets of the insert 125 can provide higher efficacy and success in prevention or correction of infant cranial shape deformities. The customized set of the inserts 125 can be based on each individual infant.

In an embodiment, the customized set of the inserts 125 can be generated based on obtained images of the infant's head. The images can be, for example, two-dimensional (2D) photogrammetry, three-dimensional (3D) photogrammetry, laser surface scans, computed tomography (CT), magnetic resonance imaging (MRI), any modality that can provide data by which an accurate rendition of the head shape can be generated, or any combination thereof. The obtained images can be analyzed and used to generate the insert 125 via, for example, additive manufacturing, injection molding, CNC machining, or vacuum casting, among others. To this end, a material of the insert 125 can be, for example, thermoplastic polyurethane, ABS, PLA, nylon, a metal, an alloy, a composite, a resin, or other materials. Further, the insert 125 can include a layer of cushion disposed on a surface of the insert 125 to improve comfort of the infant disposed thereon. For example, a material of the cushion can be a foam, polymer, or other materials.

For example, the caregiver of a newborn infant can use an imaging device, such as the caregiver's smartphone, to obtain multiple images of the newborn infant's head at various angles and orientations. The obtained images can optionally be calibrated or referenced using a known reference measurement device, or the caregiver can obtain the images while including a known reference measurement device in the image with the infant's head. In any case, the obtained images can be analyzed, either separately or in combination, to determine a shape of the newborn infant's head. This can include dimensions, curvatures, angles, and any deformities. The obtained images, and optionally a generated 3D representation based on the obtained and analyzed images, can be reviewed by a technician or skilled professional for accuracy and subsequently modified if needed.

The determined shape of the newborn infant's head can be used to determine a deviation from a target head shape for a corresponding newborn infant. The corresponding newborn infant can have similar head dimensions (circumference, width, length, depth, etc.), overall weight, overall length, etc. Based on the deviation between the newborn infant's head and the target head shape, the interior shape of the bowl 135 for the first insert of the set of the inserts 125 can be determined and the first insert can be produced. Additionally or alternatively, the second insert (and the third insert and the fourth insert) of the set of the inserts 125 can be produced based on a predicted head shape progression. In an embodiment, the interior shape of the bowl 135 can be determined by obtaining the images of the head of the infant, determining a head shape of the infant based on the obtained images, determining the deviation between the determined head shape of the infant and a head shape for a reference infant, and determining the interior shape that mitigates the deviation between the determined head shape of the infant and the head shape for the reference infant.

Upon reaching the next developmental stage, such as the infant turning 1-month old, the caregiver can obtain updated images of the infant's head at the various angles and orientations (either the same or different angles and orientations). The updated images can be analyzed, either separately or in combination, to determine a shape of the 1-month-old infant's head. The determined shape of the 1-month-old infant's head can be used to determine a deviation from a target head shape for a corresponding 1-month-old infant. The determined head shape of the 1-month-old infant can also be used to determine whether the second insert of the set of the inserts 125 (that was optionally already produced and provided to the caregiver with the first insert of the set of the inserts 125) is still acceptable for use with the 1-month-old infant. That is, an updated interior shape for the second insert of the set of the inserts 125 can be determined and whether the already produced second insert has the interior shape similar to the updated second insert interior shape. When the second insert of the set of the inserts 125 already produced and provided to the caregiver with the first insert of the set of the inserts 125 is not acceptable for use (the interior shapes are too different), the updated second insert of the set of the inserts 125 that is more acceptable based on the obtained images of the 1-month-old infant's head can be produced and provided to the caregiver. Additionally or alternatively, the third insert (and the fourth insert) of the set of the inserts 125 can be produced based on an updated predicted head shape progression.

To achieve the aforementioned non-limiting example, the caregiver's smartphone can include a software application or the like for prompting the caregiver to obtain the images of the infant's head. The software application can additionally facilitate analysis and/or transmission of the obtained images to a server for further analysis and/or for producing the corresponding inserts. For example, the server can receive the obtained images, analyze the obtained images, determine the infant's head shape, and use the infant's head shape data to generate (produce) the corresponding insert 125, such as via 3D printing. Full analysis of the obtained images at the server can allow for greater processing power and/or reduce the processing power needed by the caregiver's smartphone (or other local device). The corresponding insert 125 can then be provided to the caregiver. For example, when the already provided second insert of the set of the inserts 125 is not acceptable, the software application and send a notification to the caregiver to not swap in the already provided second insert and to wait for the updated second insert that should be swapped in instead. Additionally or alternatively, the caregiver can transfer the dimension data corresponding to the first (or second, or third, or further) insert, such as via USB, to a manufacturing device (e.g., 3D printer) and produce the target insert. Advantageously, this can be performed locally for rapid adjustments to the inserts.

In an embodiment, obtaining the images and generating the inserts can be performed in a clinical setting. For example, premature infants placed in the neonatal intensive care unit (NICU) can often spend most of their time in a supine position with little neck muscle development to change positioning of their head, leading to plagiocephaly. To prevent this, at most, in some settings a soft pad (e.g., a donut gel pad or pillow) is placed under the premature infant's head. However, these are almost all identical in shape and dimensions, and do not provide customized head shape deformity prevention or correction. Therefore, the customized set of the inserts 125 can be generated via caregivers in the NICU obtaining periodic images of the premature infant's head and/or via any needed medical imaging performed on the premature infant (e.g., CT, MRI, etc.). In an embodiment, the medical imaging images can be automatically reviewed, such as when the images are automatically transmitted to the server. The premature infant can be arranged on the base 101 in any assistive medical equipment and an updated insert 125 can periodically be swapped in as the premature infant grows to prevent or correct for any head shape deformities. Other imaging modalities can be contemplated, such as 3D laser scanning (e.g., LIDAR) and photogrammetry mentioned previously.

In an embodiment, the flange 130 of the insert 125 can be elongated and angled and essentially form the main surface 105 as part of the insert 125 (and rest on the main surface 105). Similar to the main surface 105, the elongated flange 130 can be configured to support the body of the infant disposed thereon.

In an embodiment, the insert 125 can include the elongated flange 130, as well as supports and/or trusses similar to the underside of the base 101, and the insert 125 can include a skirt similar to the skirt 115 of the base 101. That is, the insert 125 itself can be configured to entirely support the infant by itself without the base 101, and can be considered a standalone platform. In such a scenario, the standalone platform need not be inserted into any base.

In other words, the standalone platform can include a bowl (similar to the bowl 135) disposed at a first end of the standalone platform and a main surface (similar to the main surface 105 and the elongated flange 130) extending outward from an edge of the bowl to form a central portion and a second end of the standalone platform. The main surface of the standalone platform can be configured to support the infant disposed thereon and have a predetermined incline angle. The standalone platform can include a skirt (similar to the skirt 115) flanking the main surface and configured to elevate the main surface off a ground plane by a height via the skirt. Here, the platform and the skirt are formed as a single, unitary piece. Further, the bowl of the standalone platform is configured to receive a head of the infant disposed therein, an interior shape of the bowl of the standalone platform is configured to adjust a shape of the head of the infant, and the interior shape of the bowl of the standalone platform is based on a determined target head shape for the infant.

Similarly, the standalone platform can also be customized and replaced as the infant grows. Notably, the standalone platform can be customized to adjust not only for growth of the infant's head, but also the infant's body. During the newborn stage, the size of the infant's body can be smaller, and therefore a correspondingly smaller standalone platform can be generated with a smaller elongated flange 130 (main surface) that can save space (smaller footprint). By the same token, the infant can also grow quickly and become too large for the universal base 101 previously described, and therefore the standalone platform can be generated based on the large infant with a correspondingly larger elongated flange 130 (main surface) to accommodate the above-average sized infant for the corresponding growth stage. Again, techniques described previously to image the infant's head can be used to image the infant's body and similarly generate the updated standalone platform with the correspondingly updated elongated flange 130 (main surface). It may be appreciated that the elongated flange 130 (main surface) need not be updated along with the standalone platform, and a universal size and shape for the elongated flange 130 (main surface) can still be used.

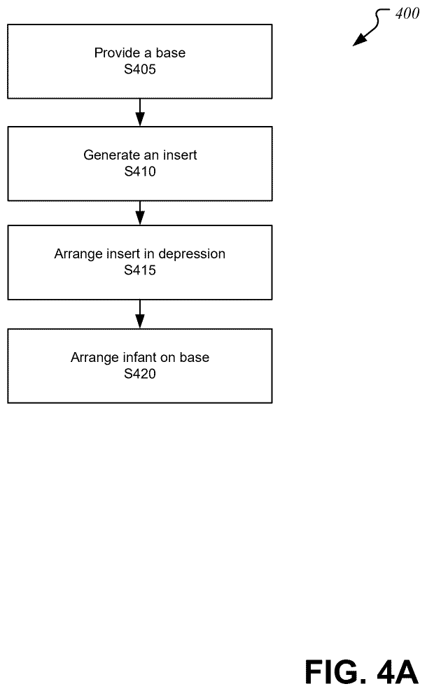

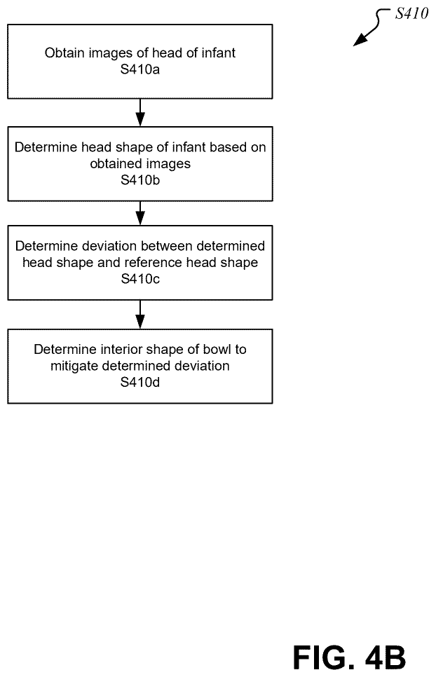

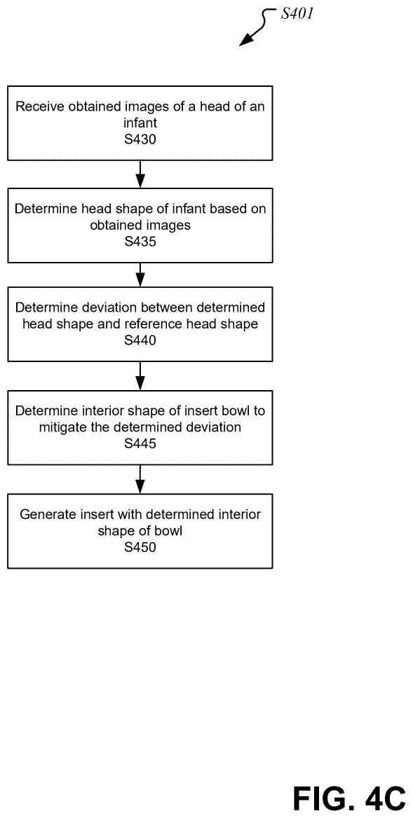

FIG. 4A is a flow chart for a method 400 of preventing and correcting cranial shape deformities, according to an embodiment of the present disclosure.

In an embodiment, step S405 is providing a base including a main surface elevated off a ground plane by a height via a skirt flanking the main surface, the main surface including a depression disposed at a first end of the main surface, the depression defined by an opening in the main surface, the main surface configured to support an infant disposed thereon, the depression configured to receive a head of the infant disposed therein.

In an embodiment, step S410 is generating an insert, the insert including a bowl and a flange surrounding the bowl, the flange extending outwards from an edge of the bowl, the depression of the base configured to receive the insert inserted therein, the flange configured to rest on an edge of the depression, the bowl of the insert configured to receive the head of the infant disposed therein, an interior shape of the bowl configured to adjust a shape of the head of the infant.

In an embodiment, step S415 is arranging the insert in the depression.

In an embodiment, step S420 is arranging the infant on the base, a body of the infant being disposed on the main surface and a head of the infant being disposed in the insert.

FIG. 4B is a flow chart for a sub-method describing step S410, according to an embodiment of the present disclosure.

In an embodiment, step S410a is obtaining the images of the head of the infant.

In an embodiment, step S410b is determining a head shape of the infant based on the obtained images.

In an embodiment, step S410c is determining a deviation between the determined head shape of the infant and a reference head shape for a reference infant.

In an embodiment, step S410d is determining the interior shape of the bowl that mitigates the deviation between the determined head shape of the infant and the reference head shape for the reference infant.

FIG. 4C is a flow chart for a method 401 of preventing and correcting cranial shape deformities, according to an embodiment of the present disclosure. In an embodiment, method 401 can occur from the perspective of a remote server in communication with an imaging device, such as a smartphone used by the caregiver.

In an embodiment, step S430 is receiving obtained images of a head of an infant.

In an embodiment, step S435 is determining a head shape of the infant based on the obtained images.

In an embodiment, step S440 is determining a deviation between the determined head shape of the infant and a reference head shape for a reference infant.

In an embodiment, step S445 is determining an interior shape of a bowl of an insert configured to receive the head of the infant disposed therein that mitigates the deviation between the determined head shape of the infant and the reference head shape for the reference infant.

In an embodiment, step S450 is generating the insert with the determined interior shape, the insert including the bowl and a flange surrounding the bowl, the flange extending outwards from an edge of the bowl, a depression of a base configured to receive the insert inserted therein, the flange configured to rest on an edge of the depression, the interior shape of the bowl configured to adjust a shape of the head of the infant, the base including a main surface elevated off a ground plane by a height via a skirt flanking the main surface, the main surface including the depression disposed at a first end of the main surface, the depression defined by an opening in the main surface, the main surface configured to support the infant disposed thereon.

While there has been described what is presently considered to be the preferred embodiments of the invention, it will be obvious to those skilled in the art that various changed and modifications may be made therein without departing from the invention whose scope is limited only by the following claims.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

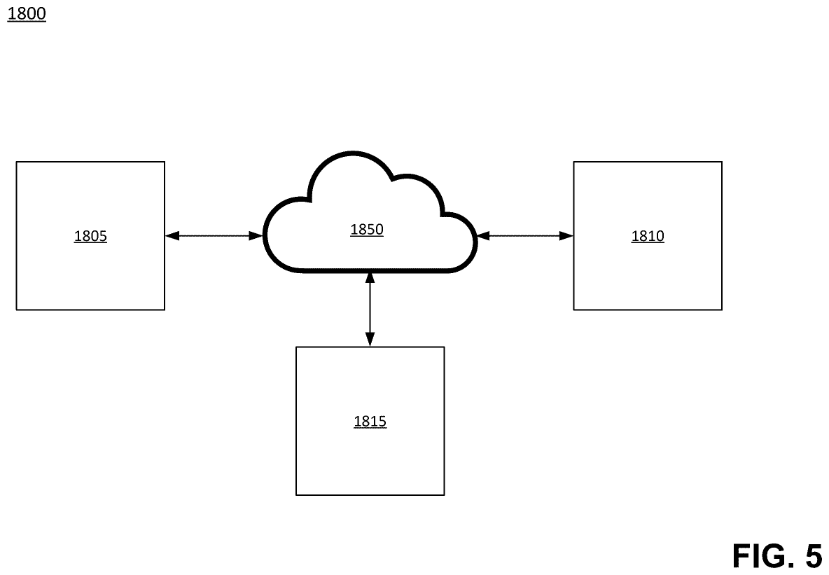

FIG. 5 is a schematic view of a system 1800, according to an embodiment of the present disclosure. In an embodiment, the system 1800 can include a first device 1805, such as the imaging device, communicatively connected to a second electronic device 1810, such as a server, via a network 1850. A third electronic device 1815, such as a computer, can be communicatively connected to the first device 1805 and the second electronic device 1810. The devices can be connected via a wired or a wireless connection. The connection between, for example, the first device 1805 and the second electronic device 1810 can be via the network 1850, wherein the network 1850 is wireless or wired. In an embodiment, the first device 1805 can be configured to obtain data from the user (of the first device 1805), such as an input relating to the infant. Notably, the first device 1805 can transmit the data over the communication network 1850 to the networked second electronic device 1810 and/or the third electronic device 1815.

In an embodiment, the first electronic device 1805 need not be communicatively coupled to the other device or the network 1850. That is, the method described herein can be run entirely on the first device 1805 using the obtained data.

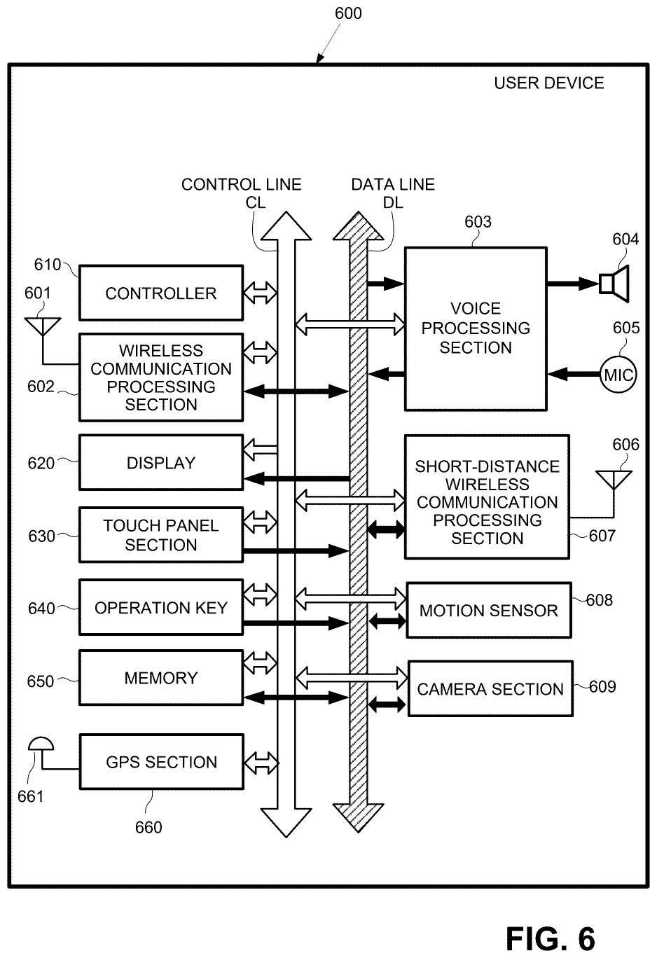

In an embodiment, the first device 1805 can include a central processing unit (CPU), among other components (discussed in more detail in FIGS. 6-8). An application can be installed or accessible on the first device 1805 for executing the methods described herein. The application can also be integrated into an operating system (OS) of the first device 1805. The first device 1805 can be or include integrated therein any electronic device such as, but not limited to, a smart-phone, a personal computer, a tablet pc, a smart-watch, a smart-television, an interactive screen, an IoT (Internet of things) device, or the like. Although the above description was discussed with respect to the first device 1805, it is to be understood that the same description applies to the other devices (1810 and 1815) of FIG. 5.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

Electronic device 600 shown in FIG. 6 can be an example of one or more of the devices shown in FIG. 5. In an embodiment, the device 600 may be a smartphone. However, the skilled artisan will appreciate that the features described herein may be adapted to be implemented on other devices (e.g., a laptop, a tablet, a server, an e-reader, a camera, a navigation device, etc.). The device 600 of FIG. 6 includes processing circuitry, as discussed above. The processing circuitry includes one or more of the elements discussed next with reference to FIG. 6. The device 600 may include other components not explicitly illustrated in FIG. 6 such as a CPU, GPU, main memory, frame buffer, etc. The device 600 includes a controller 610 and a wireless communication processor 602 connected to an antenna 601. A speaker 604 and a microphone 605 are connected to a voice processor 603.

The controller 610 may include one or more processors/processing circuitry (CPU, GPU, or other circuitry) and may control each element in the device 600 to perform functions related to communication control, audio signal processing, graphics processing, control for the audio signal processing, still and moving image processing and control, and other kinds of signal processing. The controller 610 may perform these functions by executing instructions stored in a memory 650. Alternatively, or in addition to the local storage of the memory 650, the functions may be executed using instructions stored on an external device accessed on a network or on a non-transitory computer readable medium.

The memory 650 includes but is not limited to Read Only Memory (ROM), Random Access Memory (RAM), or a memory array including a combination of volatile and non-volatile memory units. The memory 650 may be utilized as working memory by the controller 610 while executing the processes and algorithms of the present disclosure. Additionally, the memory 650 may be used for long-term storage, e.g., of image data and information related thereto.

The device 600 includes a control line CL and data line DL as internal communication bus lines. Control data to/from the controller 610 may be transmitted through the control line CL. The data line DL may be used for transmission of voice data, displayed data, etc.

The antenna 601 transmits/receives electromagnetic wave signals between base stations for performing radio-based communication, such as the various forms of cellular telephone communication. The wireless communication processor 602 controls the communication performed between the device 600 and other external devices via the antenna 601. For example, the wireless communication processor 602 may control communication between base stations for cellular phone communication.

The speaker 604 emits an audio signal corresponding to audio data supplied from the voice processor 603. The microphone 605 detects surrounding audio and converts the detected audio into an audio signal. The audio signal may then be output to the voice processor 603 for further processing. The voice processor 603 demodulates and/or decodes the audio data read from the memory 650 or audio data received by the wireless communication processor 602 and/or a short-distance wireless communication processor 607. Additionally, the voice processor 603 may decode audio signals obtained by the microphone 605.

The exemplary device 600 may also include a display 620, a touch panel 630, an operation key 640, and a short-distance communication processor 607 connected to an antenna 606. The display 620 may be an LCD, an organic electroluminescence display panel, or another display screen technology. In addition to displaying still and moving image data, the display 620 may display operational inputs, such as numbers or icons which may be used for control of the device 600. The display 620 may additionally display a GUI for a user to control aspects of the device 600 and/or other devices. Further, the display 620 may display characters and images received by the device 600 and/or stored in the memory 650 or accessed from an external device on a network. For example, the device 600 may access a network such as the Internet and display text and/or images transmitted from a Web server.

The touch panel 630 may include a physical touch panel display screen and a touch panel driver. The touch panel 630 may include one or more touch sensors for detecting an input operation on an operation surface of the touch panel display screen. The touch panel 630 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 630 may detect when the stylus approaches/contacts the operation surface of the touch panel display (similar to the case in which a finger is used for the touch operation).

In certain aspects of the present disclosure, the touch panel 630 may be disposed adjacent to the display 620 (e.g., laminated) or may be formed integrally with the display 620. For simplicity, the present disclosure assumes the touch panel 630 is formed integrally with the display 620 and therefore, examples discussed herein may describe touch operations being performed on the surface of the display 620 rather than the touch panel 630. However, the skilled artisan will appreciate that this is not limiting.

For simplicity, the present disclosure assumes the touch panel 630 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 630 may include transparent electrode touch sensors arranged in the X-Y direction on the surface of transparent sensor glass.

The touch panel driver may be included in the touch panel 630 for control processing related to the touch panel 630, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 630 may detect a position of a user's finger around an edge of the display panel 620 (e.g., gripping a protective case that surrounds the display/touch panel). Signals may be transmitted by the touch panel driver, e.g., in response to a detection of a touch operation, in response to a query from another element based on timed data exchange, etc.

The touch panel 630 and the display 620 may be surrounded by a protective casing, which may also enclose the other elements included in the device 600. In an embodiment, a position of the user's fingers on the protective casing (but not directly on the surface of the display 620) may be detected by the touch panel 630 sensors. Accordingly, the controller 610 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 610 may be configured to detect which hand is holding the device 600, based on the detected finger position. For example, the touch panel 630 sensors may detect a plurality of fingers on the left side of the device 600 (e.g., on an edge of the display 620 or on the protective casing), and detect a single finger on the right side of the device 600. In this exemplary scenario, the controller 610 may determine that the user is holding the device 600 with his/her right hand because the detected grip pattern corresponds to an expected pattern when the device 600 is held only with the right hand.

The operation key 640 may include one or more buttons or similar external control elements, which may generate an operation signal based on a detected input by the user. In addition to outputs from the touch panel 630, these operation signals may be supplied to the controller 610 for performing related processing and control. In certain aspects of the present disclosure, the processing and/or functions associated with external buttons and the like may be performed by the controller 610 in response to an input operation on the touch panel 630 display screen rather than the external button, key, etc. In this way, external buttons on the device 600 may be eliminated in lieu of performing inputs via touch operations, thereby improving watertightness.

The antenna 606 may transmit/receive electromagnetic wave signals to/from other external apparatuses, and the short-distance wireless communication processor 607 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 607.

The device 600 may include a motion sensor 608. The motion sensor 608 may detect features of motion (i.e., one or more movements) of the device 600. For example, the motion sensor 608 may include an accelerometer to detect acceleration, a gyroscope to detect angular velocity, a geomagnetic sensor to detect direction, a geo-location sensor to detect location, etc., or a combination thereof to detect motion of the device 600. In an embodiment, the motion sensor 608 may generate a detection signal that includes data representing the detected motion. For example, the motion sensor 608 may determine a number of distinct movements in a motion (e.g., from start of the series of movements to the stop, within a predetermined time interval, etc.), a number of physical shocks on the device 600 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 610, whereby further processing may be performed based on data included in the detection signal. The motion sensor 608 can work in conjunction with a Global Positioning System (GPS) section 660. The information of the present position detected by the GPS section 660 is transmitted to the controller 610. An antenna 661 is connected to the GPS section 660 for receiving and transmitting signals to and from a GPS satellite.

The device 600 may include a camera section 609, which includes a lens and shutter for capturing photographs of the surroundings around the device 600. In an embodiment, the camera section 609 captures surroundings of an opposite side of the device 600 from the user. The images of the captured photographs can be displayed on the display panel 620. A memory section saves the captured photographs. The memory section may reside within the camera section 609 or it may be part of the memory 650. The camera section 609 can be a separate feature attached to the device 600 or it can be a built-in camera feature.

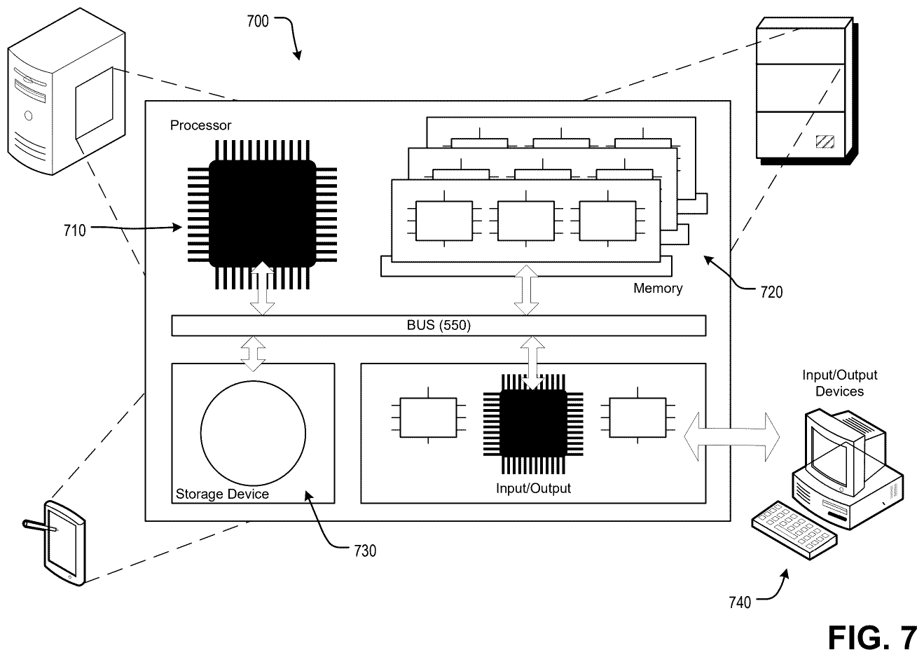

An example of a type of computer is shown in FIG. 7. The computer 700 can be used for the operations described in association with any of the computer-implement methods described previously, according to one implementation. For example, the computer 700 can be an example of devices 1805, 1815, or a server (such as device 1810). The computer 700 includes processing circuitry, as discussed above. The device 1815 may include other components not explicitly illustrated in FIG. 7 such as a CPU, GPU, main memory, frame buffer, etc. The processing circuitry includes one or more of the elements discussed next with reference to FIG. 7. In FIG. 7, the computer 700 includes a processor 710, a memory 720, a storage device 730, and an input/output device 740. Each of the components 710, 720, 730, and 740 are interconnected using a system bus 750. The processor 710 is capable of processing instructions for execution within the system 700. In one implementation, the processor 710 is a single-threaded processor. In another implementation, the processor 710 is a multi-threaded processor. The processor 710 is capable of processing instructions stored in the memory 720 or on the storage device 730 to display graphical information for a user interface on the input/output device 740.

The memory 720 stores information within the computer 700. In one implementation, the memory 720 is a computer-readable medium. In one implementation, the memory 720 is a volatile memory. In another implementation, the memory 720 is a non-volatile memory.

The storage device 730 is capable of providing mass storage for the system 700. In one implementation, the storage device 730 is a computer-readable medium. In various different implementations, the storage device 730 may be a floppy disk device, a hard disk device, an optical disk device, or a tape device.

The input/output device 740 provides input/output operations for the computer 700. In one implementation, the input/output device 740 includes a keyboard and/or pointing device. In another implementation, the input/output device 740 includes a display for displaying graphical user interfaces.

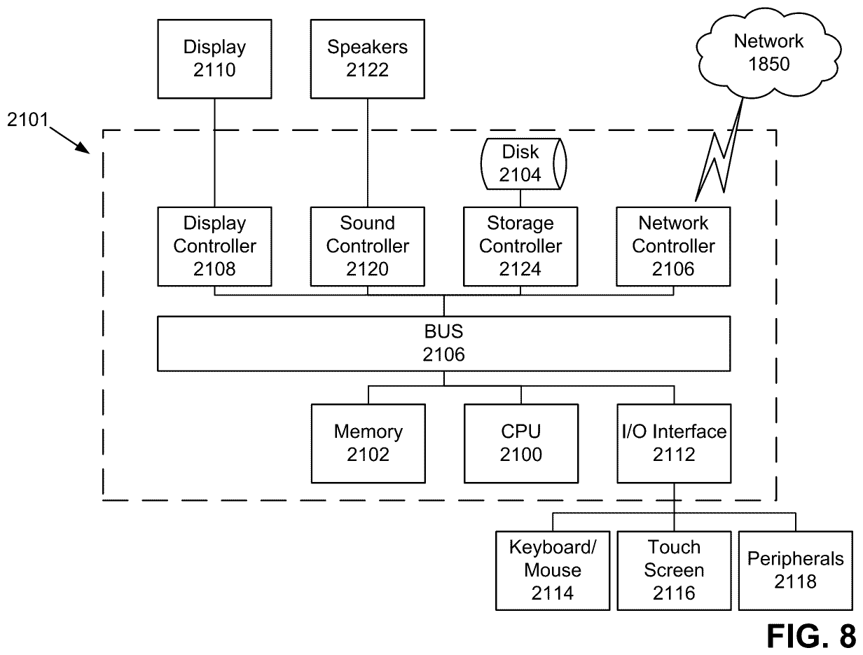

Next, a hardware description of a device 2101 according to exemplary embodiments is described with reference to FIG. 8. In FIG. 8, the device 2101, which can be the above described devices of FIG. 8, includes processing circuitry, as discussed above. The processing circuitry includes one or more of the elements discussed next with reference to FIG. 8. The device 2101 may include other components not explicitly illustrated in FIG. 8 such as a CPU, GPU, main memory, frame buffer, etc. In FIG. 8, the device 2101 includes a CPU 2100 which performs the processes described above/below. The process data and instructions may be stored in memory 2102. These processes and instructions may also be stored on a storage medium disk 2104 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device communicates, such as a server or computer.

Further, the claimed advancements may be provided as a utility application, background daemon, or component of an operating system, or combination thereof, executing in conjunction with CPU 2100 and an operating system such as Microsoft Windows, UNIX, Solaris, LINUX, Apple MAC-OS and other systems known to those skilled in the art.

The hardware elements in order to achieve the device may be realized by various circuitry elements, known to those skilled in the art. For example, CPU 2100 may be a Xenon or Core processor from Intel of America or an Opteron processor from AMD of America, or may be other processor types that would be recognized by one of ordinary skill in the art. Alternatively, the CPU 2100 may be implemented on an FPGA, ASIC, PLD or using discrete logic circuits, as one of ordinary skill in the art would recognize. Further, CPU 2100 may be implemented as multiple processors cooperatively working in parallel to perform the instructions of the processes described above.

The device 2101 in FIG. 8 also includes a network controller 2106, such as an Intel Ethernet PRO network interface card from Intel Corporation of America, for interfacing with the network 1850 (also shown in FIG. 6), and to communicate with the other devices of FIG. 6. As can be appreciated, the network 1850 can be a public network, such as the Internet, or a private network such as an LAN or WAN network, or any combination thereof and can also include PSTN or ISDN sub-networks. The network 1850 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device further includes a display controller 2108, such as a NVIDIA GeForce GTX or Quadro graphics adaptor from NVIDIA Corporation of America for interfacing with display 2110, such as an LCD monitor. A general purpose I/O interface 2112 interfaces with a keyboard and/or mouse 2114 as well as a touch screen panel 2116 on or separate from display 2110. General purpose I/O interface also connects to a variety of peripherals 2118 including printers and scanners.

A sound controller 2120 is also provided in the device to interface with speakers/microphone 2122 thereby providing sounds and/or music.

The general-purpose storage controller 2124 connects the storage medium disk 2104 with communication bus 2126, which may be an ISA, EISA, VESA, PCI, or similar, for interconnecting all of the components of the device. A description of the general features and functionality of the display 2110, keyboard and/or mouse 2114, as well as the display controller 2108, storage controller 2124, network controller 2106, sound controller 2120, and general purpose I/O interface 2112 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:
(1) An infant orthotic system, comprising: a base including a main surface elevated off a ground plane by a height via a skirt flanking the main surface, the main surface including a depression disposed at a first end of the main surface, the depression defined by an opening in the main surface, the main surface configured to support the infant disposed thereon, the depression configured to receive a head of the infant disposed therein; and an insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant.
(2) The system of (1), wherein the main surface includes an inset portion disposed at the first end of the main surface, the inset portion being defined by a recessed area in the main surface, the depression is disposed in the inset portion, and the insert is configured to be inserted into the inset portion.
(3) The system of either (1) or (2), wherein the inset portion includes a shape, and the flange of the insert forms a corresponding shape to the shape of the inset portion around the insert.
(4) The system of any one of (1) to (3), wherein when the insert is inserted in the depression in the inset portion, the flange of the insert rests on the recessed area defining the inset portion.
(5) The system of any one of (1) to (4), wherein when the insert is inserted in the depression in the inset portion, a top surface of the flange is flush with the main surface.
(6) The system of any one of (1) to (5), wherein the interior shape of the concave portion is based on a determined target head shape for the infant.
(7) The system of any one of (1) to (6), wherein the target head shape for the infant is determined based on obtained images of the head of the infant.
(8) The system of any one of (1) to (7), wherein the interior shape of the concave portion is determined by obtaining the images of the head of the infant, determining a head shape of the infant based on the obtained images, determining a deviation between the determined head shape of the infant and a reference head shape for a reference infant, and determining the interior shape of the concave portion that mitigates the deviation between the determined head shape of the infant and the reference head shape for the reference infant.
(9) The system of any one of (1) to (8), further comprising a second insert, the second insert having a second concave portion and a second flange, an interior shape of the second concave portion being based on a predicted target head shape of the infant, a shape and dimensions of the second flange being the same as the flange of the concave portion of the insert.
(10) The system of any one of (1) to (9), wherein the predicted target head shape is determined by obtaining images of the head of the infant, determining a head shape of the infant based on the obtained images, determining a deviation between the determined head shape of the infant and a reference head shape for a reference infant at a next developmental stage, and determining the interior shape of the second concave portion that mitigates the deviation between the determined head shape of the infant and the predicted target head shape for the reference infant at the next developmental stage.
(11) A method for preventing and correcting cranial shape deformities, the method including providing a base including a main surface elevated off a ground plane by a height via a skirt flanking the main surface, the main surface including a depression disposed at a first end of the main surface, the depression defined by an opening in the main surface, the main surface configured to support an infant disposed thereon, the depression configured to receive a head of the infant disposed therein; generating an insert, the insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant; arranging the insert in the depression; and arranging the infant on the base, a body of the infant being disposed on the main surface and a head of the infant being disposed in the insert.
(12) The method of (11), wherein the main surface includes an inset portion disposed at the first end of the main surface, the inset portion being defined by a recessed area in the main surface, the depression is disposed in the inset portion, and the insert is configured to be inserted into the inset portion.
(13) The method of either (11) or (12), wherein the inset portion includes a shape, and the flange of the insert forms a corresponding shape to the shape of the inset portion around the insert.
(14) The method of any one of (1) to (13), wherein when the insert is inserted in the depression in the inset portion, the flange of the insert rests on the recessed area defining the inset portion.
(15) The method of any one of (1) to (14), wherein when the insert is inserted in the depression in the inset portion, a top surface of the flange is flush with the main surface.
(16) The method of any one of (1) to (15), wherein the interior shape of the concave portion is based on a determined target head shape for the infant.
(17) The method of any one of (1) to (16), wherein the target head shape for the infant is determined based on obtained images of the head of the infant.
(18) The method of any one of (1) to (17), wherein the generating the insert further comprises obtaining the images of the head of the infant; determining a head shape of the infant based on the obtained images; determining a deviation between the determined head shape of the infant and a head shape for a reference infant; and determining the interior shape of the concave portion that mitigates the deviation between the determined head shape of the infant and the head shape for the reference infant.
(19) The method of any one of (1) to (18), further comprising: providing a second insert, the second insert having a second concave portion and a second flange, an interior shape of the second concave portion being based on a predicted target head shape of the infant, a shape and dimensions of the second flange being the same as the flange of the concave portion of the insert, the predicted target head shape being determined by obtaining images of the head of the infant; determining a head shape of the infant based on the obtained images; determining a deviation between the determined head shape of the infant and a head shape for a reference infant at a next developmental stage; and determining the interior shape of the second concave portion that mitigates the deviation between the determined head shape of the infant and the predicted target head shape for the reference infant at the next developmental stage.
(20) An infant orthotic device, including: a platform, either formed as a shell or a solid block, that has a concave portion disposed at a first end of the platform and a main surface extending outward from an edge of the concave portion to form a central portion and a second end of the platform, the main surface configured to support the infant disposed thereon, the main surface having a predetermined incline angle; and a skirt flanking the main surface and configured to elevate the main surface off a ground plane by a height via the skirt, wherein the platform and the skirt are formed as a single, unitary piece, the concave portion is configured to receive a head of the infant disposed therein, an interior shape of the concave portion is configured to adjust a shape of the head of the infant, and the interior shape of the concave portion is based on a determined target head shape for the infant.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. An infant orthotic system, comprising:
a base including a main surface elevated off a ground plane by a height via a skirt flanking the main surface, the main surface including a depression disposed at a first end of the main surface, the depression defined by an opening in the main surface, the main surface configured to support the infant disposed thereon, the depression configured to receive a head of the infant disposed therein; and
an insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant.

2. The system of claim 1, wherein
the main surface includes an inset portion disposed at the first end of the main surface, the inset portion being defined by a recessed area in the main surface,
the depression is disposed in the inset portion, and
the insert is configured to be inserted into the inset portion.

3. The system of claim 2, wherein
the inset portion includes a shape, and
the flange of the insert forms a corresponding shape to the shape of the inset portion around the insert.

4. The system of claim 3, wherein when the insert is inserted in the depression in the inset portion, the flange of the insert rests on the recessed area defining the inset portion.

5. The system of claim 3, wherein when the insert is inserted in the depression in the inset portion, a top surface of the flange is flush with the main surface.

6. The system of claim 1, wherein the interior shape of the concave portion is based on a determined target head shape for the infant.

7. The system of claim 6, wherein the target head shape for the infant is determined based on obtained images of the head of the infant.

8. The system of claim 7, wherein the interior shape of the concave portion is determined by
obtaining the images of the head of the infant,
determining a head shape of the infant based on the obtained images,
determining a deviation between the determined head shape of the infant and a reference head shape for a reference infant, and
determining the interior shape of the concave portion that mitigates the deviation between the determined head shape of the infant and the reference head shape for the reference infant.

9. The system of claim 6, further comprising a second insert, the second insert having a second concave portion and a second flange, an interior shape of the second concave portion being based on a predicted target head shape of the infant, a shape and dimensions of the second flange being the same as the flange of the concave portion of the insert.

10. The system of claim 9, wherein the predicted target head shape is determined by
obtaining images of the head of the infant,
determining a head shape of the infant based on the obtained images,
determining a deviation between the determined head shape of the infant and a reference head shape for a reference infant at a next developmental stage, and
determining the interior shape of the second concave portion that mitigates the deviation between the determined head shape of the infant and the predicted target head shape for the reference infant at the next developmental stage.

11. A method for preventing and correcting cranial shape deformities, the method comprising:
providing a base including a main surface elevated off a ground plane by a height via a skirt flanking the main surface, the main surface including a depression disposed at a first end of the main surface, the depression defined by an opening in the main surface, the main surface configured to support an infant disposed thereon, the depression configured to receive a head of the infant disposed therein;
generating an insert, the insert including a concave portion and a flange surrounding the concave portion, the flange extending outwards from an edge of the concave portion, the depression of the base configured to receive the insert inserted therein, the flange configured to rest on an edge of the depression, the concave portion of the insert configured to receive the head of the infant disposed therein, an interior shape of the concave portion configured to adjust a shape of the head of the infant;
arranging the insert in the depression; and
arranging the infant on the base, a body of the infant being disposed on the main surface and a head of the infant being disposed in the insert.

12. The method of claim 11, wherein
the main surface includes an inset portion disposed at the first end of the main surface, the inset portion being defined by a recessed area in the main surface,
the depression is disposed in the inset portion, and
the insert is configured to be inserted into the inset portion.

13. The method of claim 12, wherein
the inset portion includes a shape, and
the flange of the insert forms a corresponding shape to the shape of the inset portion around the insert.

14. The method of claim 13, wherein when the insert is inserted in the depression in the inset portion, the flange of the insert rests on the recessed area defining the inset portion.

15. The method of claim 13, wherein when the insert is inserted in the depression in the inset portion, a top surface of the flange is flush with the main surface.

16. The method of claim 11, wherein the interior shape of the concave portion is based on a determined target head shape for the infant.

17. The method of claim 16, wherein the target head shape for the infant is determined based on obtained images of the head of the infant.

18. The method of claim 17, wherein the generating the insert further comprises
obtaining the images of the head of the infant;
determining a head shape of the infant based on the obtained images;
determining a deviation between the determined head shape of the infant and a head shape for a reference infant; and
determining the interior shape of the concave portion that mitigates the deviation between the determined head shape of the infant and the head shape for the reference infant.

19. The method of claim 16, further comprising:
providing a second insert, the second insert having a second concave portion and a second flange, an interior shape of the second concave portion being based on a predicted target head shape of the infant, a shape and dimensions of the second flange being the same as the flange of the concave portion of the insert, the predicted target head shape being determined by
obtaining images of the head of the infant;
determining a head shape of the infant based on the obtained images;
determining a deviation between the determined head shape of the infant and a reference head shape for a reference infant at a next developmental stage; and
determining the interior shape of the second concave portion that mitigates the deviation between the determined head shape of the infant and the predicted target head shape for the reference infant at the next developmental stage.

20. An infant orthotic device, comprising:
a platform, either formed as a shell or a solid block, that has a concave portion disposed at a first end of the platform and a main surface extending outward from an edge of the concave portion to form a central portion and a second end of the platform, the main surface configured to support the infant disposed thereon, the main surface having a predetermined incline angle; and
a skirt flanking the main surface and configured to elevate the main surface off a ground plane by a height via the skirt, wherein
the platform and the skirt are formed as a single, unitary piece,
the concave portion is configured to receive a head of the infant disposed therein,
an interior shape of the concave portion is configured to adjust a shape of the head of the infant, and
the interior shape of the concave portion is based on a determined target head shape for the infant.
